(19) United States
(12) Patent Application Publication
(10) Pub. No.: US 2016/0331219 A1
(43) Pub. Date: Nov. 17, 2016

(54) ENDOSCOPY SERVICE SUPPORT APPARATUS

(71) Applicant: OLYMPUS CORPORATION, Tokyo (JP)

(72) Inventors: Yoshiko TAKAHASHI, Tokyo (JP); Masakazu OMOTO, Tokyo (JP); Yoshihide ENDO, Tokyo (JP)

(73) Assignee: OLYMPUS CORPORATION, Tokyo (JP)

(21) Appl. No.: 15/221,796

(22) Filed: Jul. 28, 2016

Related U.S. Application Data
(63) Continuation of application No. PCT/JP2015/063456, filed on May 11, 2015.

(30) Foreign Application Priority Data
Aug. 1, 2014 (JP) 2014-157397

(51) Int. Cl. A61B 1/12; G05B 19/042; G06F 19/00; A61B 1/04; B08B 9/027

(52) U.S. Cl. CPC A61B 1/121; A61B 1/04; B08B 9/027; G06F 19/3412; G05B 19/042; G05B 2219/45118

(57) ABSTRACT

A retrieval unit retrieves examination information: that includes the identification information on an endoscope corresponding to the identification information on an endoscope included in cleaning information; and in which an examination end time included in the examination information is prior to a cleaning start time included in the cleaning information. An association candidate determination unit determines, of examination information that satisfy a retrieval condition, examination information including an examination end time closest to the cleaning start time as a candidate for the examination information that should be associated with the cleaning information. A time conversion unit converts a cleaning start time of standard time included in the cleaning information into a cleaning start time of summer time. A retrieval unit re-retrieves examination information by using the converted cleaning start time of summer time.

### CROSS-REFERENCE TO RELATED APPLICATIONS

This application is a continuation of and claims priority to PCT/JP2015/063456 filed May 11, 2015, which claims the benefit of and priority from Japanese Patent Application No. 2014-157397 filed Aug. 1, 2014, the entire contents of these applications are incorporated herein by reference.

### BACKGROUND OF THE INVENTION

1. Field of the Invention

The present invention relates to an endoscopy service support apparatus for supporting endoscopy services.

2. Description of the Related Art

Some endoscopy service support systems have a function in which examination information and cleaning information are associated with each other by checking the last imaging time, at which the inside of the body of a patient has been imaged with an endoscope (hereinafter, simply referred to as a scope), and a cleaning start time of a scope. If there is a lag between the time of a system and that of a cleaning apparatus before the association, the association between examination information and cleaning information may not be achieved. In order to avoid this, time synchronization is performed between the system and the cleaning apparatus.

Even if time synchronization is performed between the system and the cleaning apparatus, a cleaning start time becomes earlier than the last imaging time, when the time of the system is changed from summer time to the normal standard time, whereby a mismatch may be caused in the association. Once a mismatch is caused, mismatches may also be caused in the association for the subsequent examinations.

Because it becomes falsification of data to convert, for preventing this, the last imaging time of summer time into that of standard time or to convert the cleaning start time of standard time into that of summer time, such a conversion cannot be performed. Accordingly, when a mismatch is caused in the association, users manually create a new cleaning order, so that a scope that has been used in an examination and a scope to be cleaned are associated with each other. In this case, an operation becomes necessary, in which, after an investigation is performed into which examination causes the mismatch, the association is corrected, which makes operations by a user complicated.

### SUMMARY OF THE INVENTION

In order to solve the aforementioned problems, an endoscopy service support apparatus according to an embodiment of the present invention comprises: an examination information storage unit that stores examination information acquired from an endoscope system; and a control unit that retrieves, when receiving cleaning information from a cleaning apparatus for cleaning an endoscope, examination information that should be associated with the cleaning information, based on a cleaning start time included in the cleaning information and an examination end time included in examination information stored in the examination information storage unit. The control unit includes: a retrieval unit that retrieves examination information in which identification information on an endoscope, corresponding to the identification information on an endoscope included in the cleaning information, is included, and in which the examination end time included in the examination information is prior to the cleaning start time included in the cleaning information; a candidate determination unit that determines, of examination information that satisfy a retrieval condition, examination information including an examination end time closest to the cleaning start time as a candidate for examination information that should be associated with the cleaning information; and a time conversion unit that converts, when examination information that satisfies the retrieval condition is not present and when the cleaning start time is a time included in a period of time corresponding to the time difference between summer time and the subsequent standard time, the cleaning start time of standard time included in the cleaning information into a cleaning start time of summer time. The retrieval unit re-retrieves examination information by using the converted cleaning start time of summer time.

Arbitrary combinations of the aforementioned constituting elements and implementations of the invention in the form of methods, apparatuses, systems, recording media, computer programs and so forth may also be effective as additional modes of the present invention.

### BRIEF DESCRIPTION OF THE DRAWINGS

Embodiments will now be described, byway of example only, with reference to the accompanying drawings, which are meant to be exemplary, not limiting, and wherein like elements are numbered alike in several figures, in which:

FIG. 1 is a view illustrating a configuration of an endoscopy service support system according to an embodiment of the present invention;

FIG. 2 is a view illustrating an internal configuration of an endoscopy service support apparatus in FIG. 1;

FIG. 3 is a view illustrating internal configurations of a terminal apparatus and a cleaning apparatus in FIG. 1;

FIG. 4 is a view illustrating a basic concept of a method of associating examination information and cleaning information with each other;

FIG. 5 is a table in which operations, generated at around 2:00 AM on the day when summer time is changed to standard time, are written in a time-series manner;

FIG. 6 is a table showing a situation in which association with the examination information including the last imaging time of 1:55 AM can be made by converting a cleaning start time of 1:15 AM, on the day when summer time is changed to standard time, into a cleaning start time of 2:15 AM;

FIGS. 7A to 7D are tables each showing a display example in which examination information including the last imaging time of summer time and cleaning information including a cleaning start time of standard time are displayed on a screen;

FIG. 8 is a flowchart illustrating a flow of processing according to an embodiment of the invention, in which cleaning information and examination information are associated with each other; and

FIGS. 9A and 9B are tables showing examples of the examination information and the cleaning information that have been associated with each other, respectively.

### DETAILED DESCRIPTION OF THE INVENTION

The invention will now be described by reference to the preferred embodiments. This does not intend to limit the scope of the present invention, but to exemplify the invention.

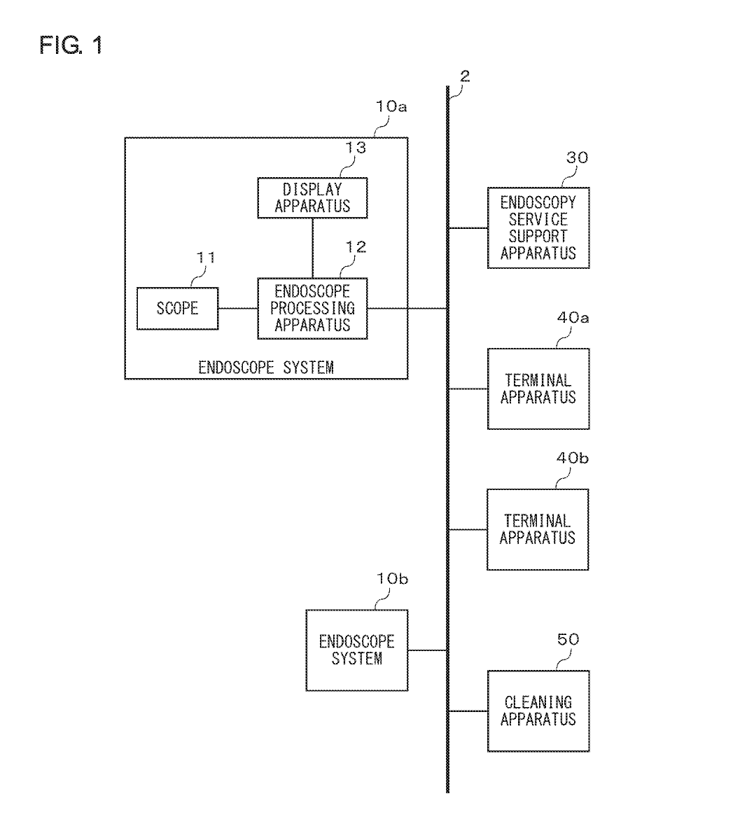

FIG. 1 is a view illustrating a configuration of an endoscopy service support system 1 according to an embodiment of the present invention. The endoscopy service support system 1 is one that is installed in an endoscopic department in order to support endoscopy services. The endoscopy service support system 1 comprises: a plurality of endoscope systems 10a and 10b (hereinafter, referred to as an “endoscope system 10”, when they are not particularly distinguished from each other); an endoscopy service support apparatus 30; a plurality of terminal apparatuses 40a and 40b (hereinafter, referred to as a “terminal apparatus 40”, when they are not particularly distinguished from each other); and a cleaning apparatus 50, these systems and apparatuses being interconnected together by a network 2, such as a LAN. Although FIG. 1 illustrates an example in which the endoscopy service support apparatus 30 and the terminal apparatus 40 forms different housings, they may be formed integrally with each other.

The endoscopy service support system 1 can be coordinated with another system in a medical facility. For example, a non-illustrated gateway device is connected to the network 2, and the endoscopy service support system 1 can be coordinated with an ordering system, an electronic medical record system, a receipt system, or the like, via the gateway device.

The endoscope system 10 includes a scope 11, an endoscope processing apparatus 12, and a display apparatus 13.

The scope 11 is inserted into the body of a patient in order to image the inside of the body at a timing when a doctor presses a release switch in the scope 11. The scope 11 includes a solid-state imaging device (e.g., a CCD image sensor, a CMD image sensor, or a CMOS image sensor) and a signal processing circuit. The solid-state imaging device converts incident light into an electrical signal, and the signal processing circuit performs signal processing, such as A/D conversion or noise removal, on the image data photoelectric-converted by the solid-state imaging device, so that the image data is output to the endoscope processing apparatus 12.

The endoscope processing apparatus 12 acquires an image captured by the scope 11. The endoscope processing apparatus 12 outputs the acquired image to the display apparatus 13, so that the image is displayed by the display apparatus 13. The image currently being captured can also be displayed in real time. Additionally, the endoscope processing apparatus 12 transmits the image acquired from the scope 11 to the endoscopy service support apparatus 30, so that it is filed in a storage unit in the endoscopy service support apparatus 30.

The endoscopy service support apparatus 30 integrally controls the whole endoscopy service support system 1. The endoscopy service support apparatus 30 is formed, for example, by a server. The terminal apparatus 40 is one to be used by users engaged in a medical institution, such as doctors and nurses, and is formed, for example, by a PC, a tablet, or a PDA. When a mobile terminal apparatus, such as a tablet or a PDA, is used, a non-illustrated access point is installed in the network 2, so that the mobile terminal apparatus is connected to the network 2 by a wireless LAN. The cleaning apparatus 50 is used for cleaning and sterilizing the scope 11 that has been used in examination or treatment.

The endoscopy service support apparatus 30 receives the endoscopic image data transmitted from the endoscope system 10, and stores it in the storage unit. The endoscopy service support apparatus 30 also stores the cleaning history of the cleaning apparatus 50 in the storage unit as cleaning information. Additionally, the endoscopy service support apparatus 30 executes various application programs relevant to endoscopy services in accordance with operations from the terminal apparatus 40 connected to the network 2, so that a service performed by a user of the terminal apparatus 40 is supported. For example, creation of an examination report by a doctor is supported by executing an examination report creation support program. When an examination report is input by a doctor operating the terminal apparatus 40, the endoscopy service support apparatus 30 receives the examination report transmitted from the terminal apparatus 40 to store it in the storage unit.

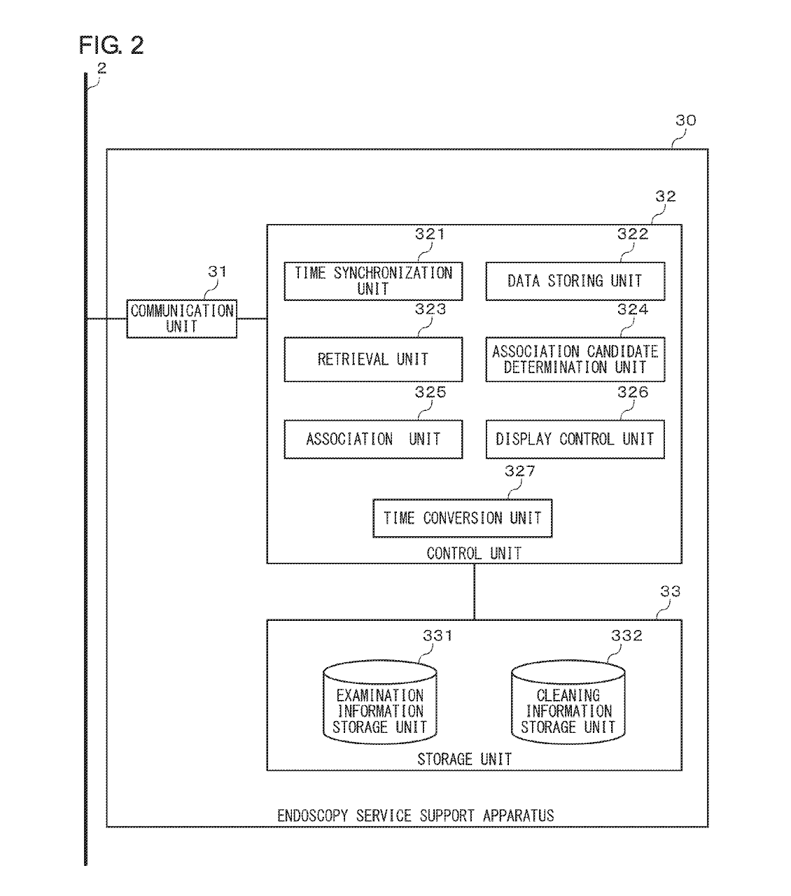

FIG. 2 is a view illustrating an internal configuration of the endoscopy service support apparatus 30 in FIG. 1. The endoscopy service support apparatus 30 includes a communication unit 31, a control unit 32, and a storage unit 33. The control unit 32 includes a time synchronization unit 321, a data storage unit 322, a retrieval unit 323, an association candidate determination unit 324, an association unit 325, a display control unit 326, and a time conversion unit 327. In the present embodiment, attention is paid to an association function (also referred to as a linking function) of examination information and cleaning information, and hence functions relevant to the association function thereof are only illustrated as the functional blocks of the control unit 32 in FIG. 2.

The functions of the control unit 32 can be achieved by cooperation of hardware resources and software resources. A processor, a ROM, a RAM, and other LSIs can be used as the hardware resources. Programs, such as an operating system and an application, can be used as the software resources.

The storage unit 33 is formed, for example, by an HDD, an SSD, or the like, and includes an examination information storage unit 331 and a cleaning information storage unit 332. In the present embodiment, attention is paid to the association function of examination information and cleaning information, and hence functions relevant to the association function thereof are only illustrated as the functional blocks of the storage unit 33 in FIG. 2.

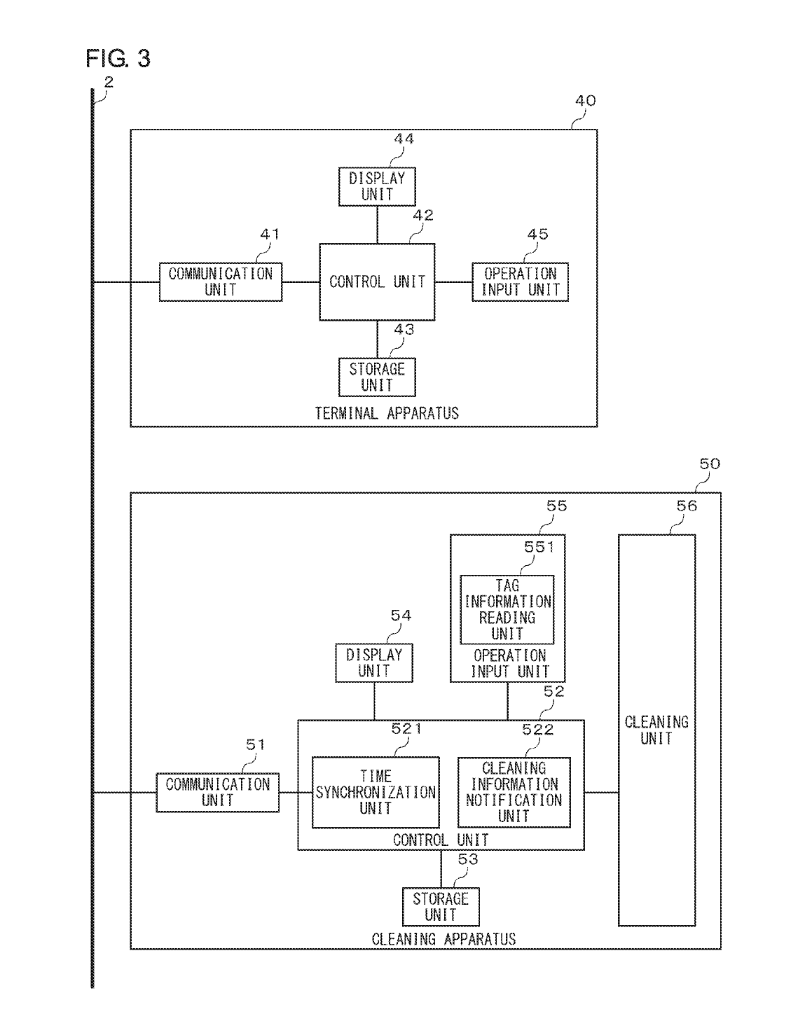

FIG. 3 is a view illustrating internal configurations of the terminal apparatus 40 and the cleaning apparatus 50 in FIG. 1. The terminal apparatus 40 includes a communication unit 41, a control unit 42, a storage unit 43, a display unit 44, and an operation input unit 45. The cleaning apparatus 50 includes a communication unit 51, a control unit 52, a storage unit 53, a display unit 54, an operation input unit 55, and a cleaning unit 56. The control unit 52 in the cleaning apparatus 50 includes a time synchronization unit 521 and a cleaning information notification unit 522. The operation input unit 55 in the cleaning apparatus 50 includes a tag information reading unit 551. Hereinafter, more specific description will be made with reference to FIGS. 2 and 3.

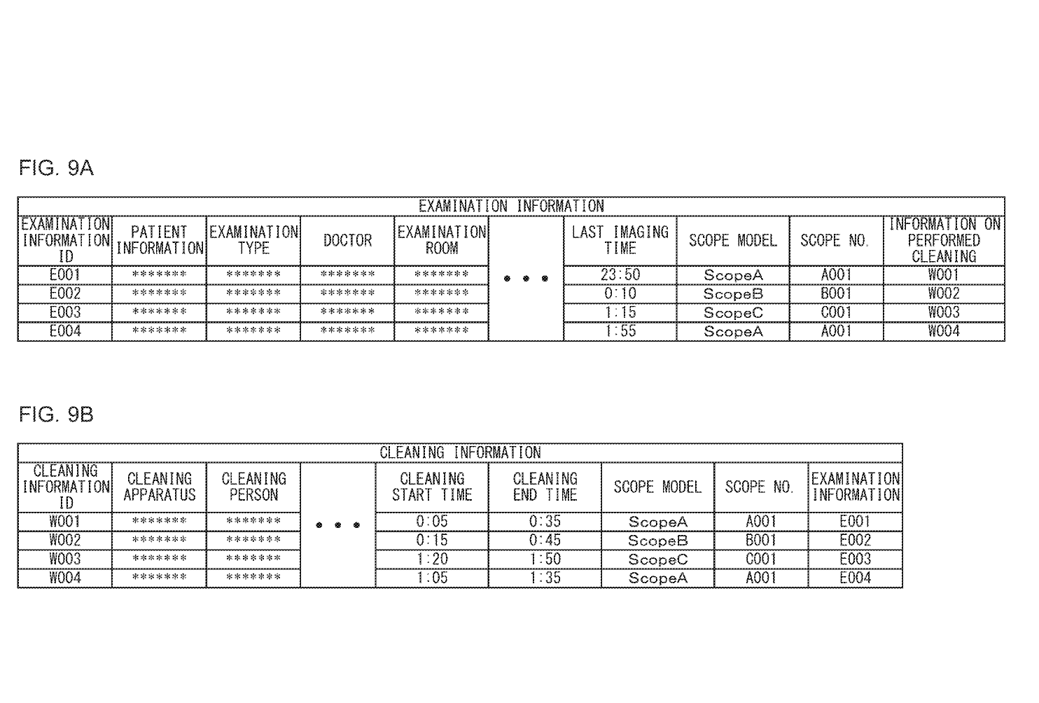

The examination information storage unit 331 in the endoscopy service support apparatus 30 stores examination information acquired from the endoscope system 10. The examination information is used for recording an objective fact regarding examination implementation, unlike an examination report including a view of a doctor. For example, patient information; information on an examination type, doctor, examination room, examination date, examination start time, examination end time, and scope; and the like are recorded as the examination information, as shown in the later-described FIG. 9A. The cleaning information storage unit 332 stores the cleaning information acquired from the cleaning apparatus 50. For example, information on a cleaning apparatus, cleaning person, cleaning start time, cleaning end time, and scope; and the like are recorded as the cleaning information, as shown in the later-described FIG. 9B.

The time synchronization unit 321 manages the system time of the endoscopy service support apparatus 30. The time synchronization unit 321 synchronizes the system time of the endoscopy service support apparatus 30 with the time of a time server on the Internet by accessing the time server via a non-illustrated gateway device. The time synchronization unit 321 also synchronizes the system time of the endoscopy service support apparatus 30 with the internal time of the cleaning apparatus 50 by accessing the cleaning apparatus 50 via the network 2. Time synchronization between the endoscopy service support apparatus 30 and the cleaning apparatus 50 is executed periodically. It is executed, for example, every minute.

The data storage unit 322 stores examination information acquired from the endoscope system 10 in the examination information storage unit 331. The data storage 322 also stores cleaning information acquired from the cleaning apparatus 50 in the cleaning information storage unit 332.

The cleaning unit 56 in the cleaning apparatus 50 is a collective term for members with which cleaning is actually performed, including a cleaning tank, antiseptic solution tank, pump, valve, hose, and the like. The time synchronization unit 521 in the control unit 52 synchronizes the time transmitted from the endoscopy service support apparatus 30 with the internal time of the cleaning apparatus 50.

Identification information for uniquely identifying self is attached to each scope 11. For example, an IC tag of RFID (Radio Frequency IDentification) type is attached to the code of the scope 11. A cleaning person causes the tag information reading unit 551 to read the identification information on the scope 11 by holding up the tag of the scope 11 over the tag information reading unit 551 in the operation input unit 55. The cleaning person also causes the tag information reading unit 551 to read his/her identification information by holding up the IC tag added to the identification card held by him/her over the tag information reading unit 551.

When cleaning of the scope 11 is started, the cleaning information notification unit 522 in the control unit 52 notifies the endoscopy service support apparatus 30 of cleaning information including: the identification information on the scope 11 and the identification information on a cleaning person, which have been read by the tag information reading unit 551; and a cleaning start time. Herein, the same cleaning information as the notified cleaning information are also stored in the storage unit 53. When the cleaning of the scope 11 is ended, the cleaning information notification unit 522 notifies the endoscopy service support apparatus 30 of cleaning information including at least a cleaning end time. Various information are displayed in the display unit 54, and, for example, a remaining cleaning time of the scope 11 is displayed.

When receiving cleaning information from the cleaning apparatus 50, the control unit 32 in the endoscopy service support apparatus 30 retrieves the examination information that should be associated with the cleaning information based on the cleaning start time included in the cleaning information and the examination end time included in the examination information to be stored in the examination information storage unit 331. The control unit 32 retrieves the examination information that should be associated with the cleaning information, each time when the control unit 32 receives, from the cleaning apparatus 50, cleaning information for notification of cleaning end including a cleaning end time. The cleaning information for notification of cleaning end including a cleaning end time is integrated with the cleaning information for notification of cleaning start including a cleaning start time, which has been previously acquired. Hereinafter, more specific description will be made.

The retrieval unit 323 retrieves examination information: that includes the identification information on the scope 11 that matches the identification information on the scope 11 included in the cleaning information to be associated with; in which the examination end time included in the examination information is prior to the cleaning start time included in the cleaning information; and that is not associated with another cleaning information. The association candidate determination unit 324 determines, of the examination information that satisfy the aforementioned retrieval conditions, the examination information including an examination end time closest to the cleaning start time as a candidate for the examination information that should be associated with the cleaning information.

The display control unit 326 causes the display unit 44 in the terminal apparatus 40 to display a screen for making a user (e.g., a nurse) select whether the candidate for the examination information, which has been determined by the association candidate determination unit 324, is to be associated with the cleaning information. When an agreement operation is performed on the operation input unit 45 in the terminal apparatus 40 by a user who has watched the screen, the association unit 325 associates the cleaning information and the examination information with each other.

When the examination information that satisfies the aforementioned retrieval conditions is not present, the association candidate determination unit 324 determines whether the cleaning start time included in the cleaning information is a time included in a period of time corresponding to the time difference between summer time and the subsequent standard time (1 hour in the U.S.A.). In the U.S.A., summer time starts on the first Sunday in April and ends on the final Sunday in October. On the first Sunday in April, 1:59:59 AM is followed by 3:00:00 AM. That is, a period of time between 2:00:00 AM and 2:59:59 AM on the first Sunday in April is skipped, and time is advanced by 1 hour. On the final Sunday in October, 1:59:59 AM is followed by 1:00:00 AM. That is, periods of time between 1:00:00 AM and 1:59:59 AM are generated two times on the final Sunday in October, and time is delayed by 1 hour.

When the cleaning start time included in the cleaning information is a time included in a period of time corresponding to the time difference between summer time and the subsequent standard time, the time conversion unit 327 converts the cleaning start time of standard time included in the cleaning information into a cleaning start time of summer time. The retrieval unit 323 re-retrieves examination information by using the converted cleaning start time of summer time. When the examination information that satisfy the aforementioned retrieval conditions are retrieved by the re-retrieval, the association candidate determination unit 324 determines examination information including an examination end time closest to the converted cleaning start time of summer time as a candidate for the examination information that should be associated with the cleaning information. When examination information that satisfies the aforementioned retrieval conditions is not retrieved even by the re-retrieval, the association candidate determination unit 324 determines the cleaning information as cleaning information on cleaning that has been performed independently of examination, which is not to be associated with examination information.

Of the imaging times when the inside of the body of a patient has been imaged by using the scope 11 used in the examination, the last imaging time is used as the aforementioned examination end time in the present embodiment. Alternatively, it can be considered that: a time when an examination end button provided in the endoscope processing apparatus 12 is pressed; a time when a patient leaves a room, the time being input in the endoscope processing apparatus 12 by a nurse; or the like, is used as the examination end time. It is rather more desirable to use such a time as a record of examination information than to use the last imaging time; however, in order to associate cleaning information and examination information with each other, it is more desirable to use the last imaging time.

In an endoscopic examination, recovery of a patient is required after the scope 11 is extracted from the body of the patient. Additionally, some doctors make explanation on symptoms to patients after the scope 11 is extracted. In order to make more correct records of examination information, it is desirable to include recovery of a patient and explanation to a patient into examination and to record the end time of the examination including them. However, the scope 11 may be subjected to cleaning immediately after being extracted from the body of a patient. When a plurality of examinations are appointed in a medical facility having a small number of the scopes 11, it is highly required to clean the scope 11 immediately. In this case, there is the possibility that the cleaning start time of the scope 11 may be prior to the time when a patient leaves a room, whereby a mismatch may be caused in associating cleaning information and examination information with each other.

Regarding this point, there is the relationship that the last imaging time of the scope 11 is always prior to the cleaning start time of the scope 11, whereby a mismatch possibly generated in the association can be suppressed. Additionally, an imaging time of the scope 11 is imparted to captured image data at a timing when a release switch in the scope 11 is turned off by a doctor. The imaging time is imparted by the scope 11, and is highly reliable. When an examination end time is input by an artificial operation, such as press of an examination end button or input by a nurse, there is the possibility that a doctor or a nurse may operate at a delayed timing, so that a lag may be generated with the actual time.

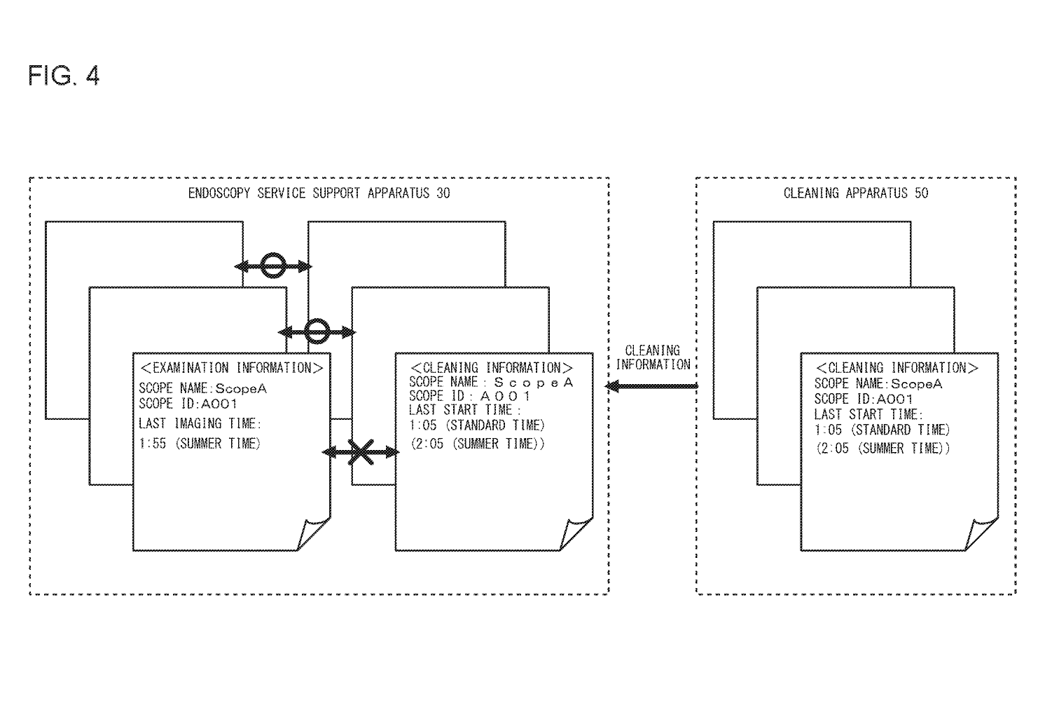

FIG. 4 is a view illustrating a basic concept of a method of associating examination information and cleaning information with each other. The scope 11 used in an endoscopic examination is necessarily required to be cleaned, and accordingly examination and cleaning are basically combined as a set. The scope 11 may be used multiple times a day, but in that case, the scope 11 is also required to be cleaned upon every examination end. The scope 11 may be exceptionally cleaned independently of examination. The scope 11 is cleaned, for example, when used for the first time after being opened, or when used after being stored for a long time without being used.

Information on the association between examination information and cleaning information are utilized for: investigating a cause of hospital infection occurrence; proving works by a medical professional when a medical malpractice problem is caused; or the like. Accordingly, it is necessary to prevent falsification of data, and an easy change is not acceptable.

The examination information and the cleaning information on one scope 11 are basically associated with each other at a ratio of 1:1, as illustrated in FIG. 4. In FIG. 4, a scope name (scope model, model number) and a scope number are used as the identification information on the scope 11. In the present embodiment, the endoscopy service support apparatus 30 receives cleaning information from the cleaning apparatus 50 each time when cleaning is ended, so that the examination information to be associated with the cleaning information is retrieved, as described above. When the aforementioned retrieval conditions are applied, a candidate for the examination information that should be associated with the cleaning information is basically one of the examination information on the latest examination in which the scope 11 having the same identification information has been used.

As illustrated in FIG. 4, however, when a cleaning start time is within a period of time of 1:00 AM to 1:59 AM of the second time (standard time) on the day when summer time is changed to standard time and when an imaging end time is within a period of time of 1:00 AM to 1:59 AM of the first time (summer time), the cleaning information and the examination information, which should be essentially associated with each other, may not be associated with each other.

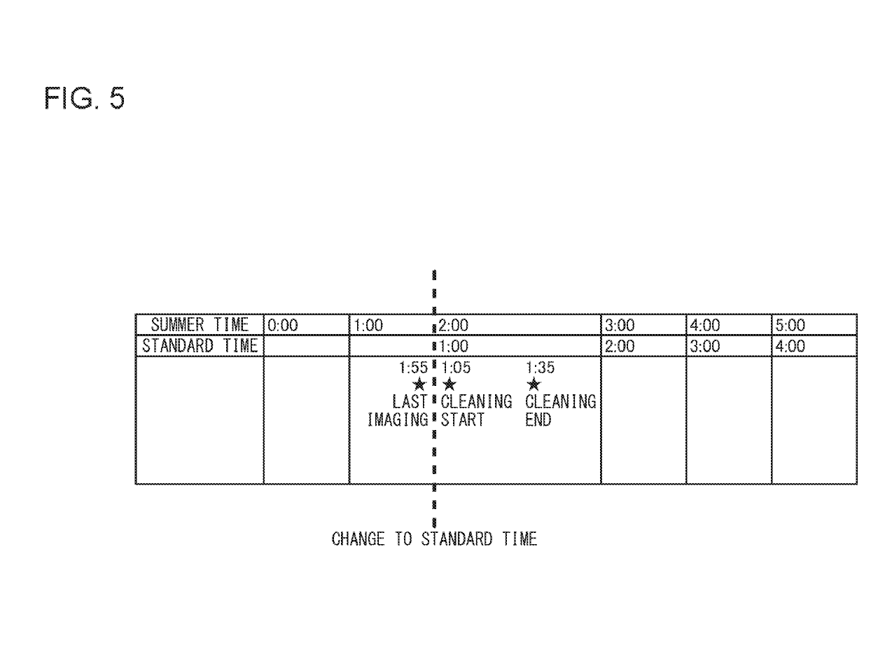

FIG. 5 is a table in which operations, generated at around 2:00 AM on the day when summer time is changed to standard time, are written in a time-series manner. FIG. 5 shows an example in which the last imaging time of the scope 11 shown in FIG. 4 is 1:55 AM of the first time (summer time) and the cleaning start time of the scope 11 is 1:05 AM of the second time (standard time). In this case, when both the notational times are compared to each other, the cleaning start time is prior, which causes an inconsistency. Both are information that should actually be associated with each other, but a situation is generated, in which they cannot be candidates for the association. As described above, when 2:00 AM on the day when summer time is changed to standard time is sandwiched between the last imaging time and a cleaning start time, an inconsistency is caused. In this case, the cleaning information is processed as single cleaning information not to be associated with examination.

When the subsequent examination using the same scope 11 is conducted and the cleaning of the scope 11 used in the examination is ended, new cleaning information is generated to be notified to the endoscopy service support apparatus 30. If the examination information on the examination is not stored yet at the time, the examination information whose last imaging time is 1:55 AM is cited as a candidate for the association with the new cleaning information. If a user erroneously permits the association between them, each of the subsequent association is shifted by one. When the shifts are corrected later, it is necessary to manually recreate cleaning orders in all of the shifted cleaning information, which requires great care.

Accordingly, a function is added in the present embodiment, in which, when a candidate, for the examination information to be associated with the cleaning information including a cleaning start time within a period of time of 1:00 AM to 1:59 AM on the day when summer time is changed to standard time, is not retrieved, re-retrieval is performed by converting the cleaning start time into a cleaning start time of summer time. That is, re-retrieval is performed by using a time obtained by adding one hour to the cleaning start time included in the cleaning information.

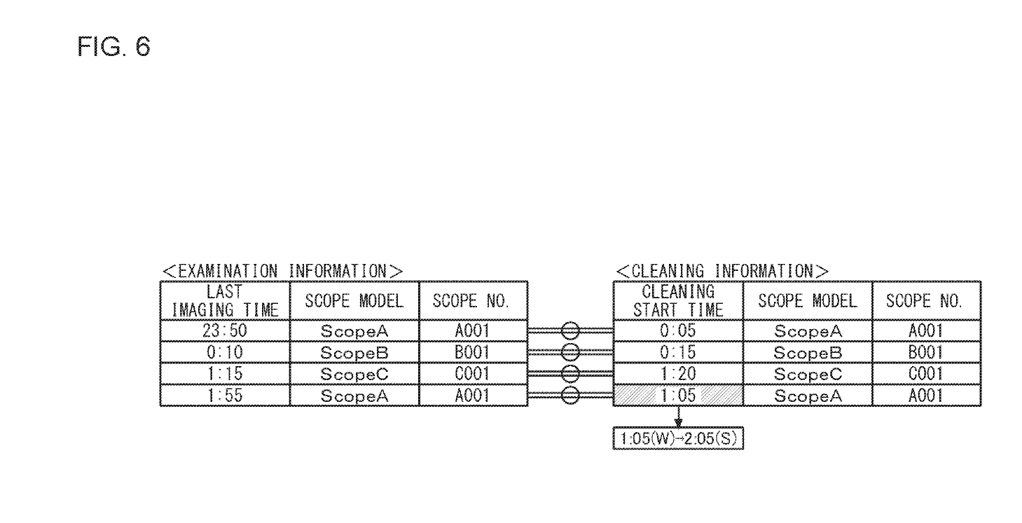

FIG. 6 is a table showing a situation in which association with the examination information including the last imaging time of 1:55 AM can be made by converting a cleaning start time of 1:15 AM, on the day when summer time is changed to standard time, into a cleaning start time of 2:15 AM.

With such conversion processing, examination information including an examination end time of summer time can be made a candidate for the association with cleaning information including a cleaning start time of standard time, even if there is an inconsistency between them. When both examination information including an examination end time of summer time and cleaning information including a cleaning start time of standard time are displayed on a screen, the display control unit 326 makes them displayed such that a user can recognize a difference in time notation between them. For example, the display control unit 326 makes a mark for the recognition of the difference displayed, classifies them by color, or makes two types of time including summer time and standard time displayed.

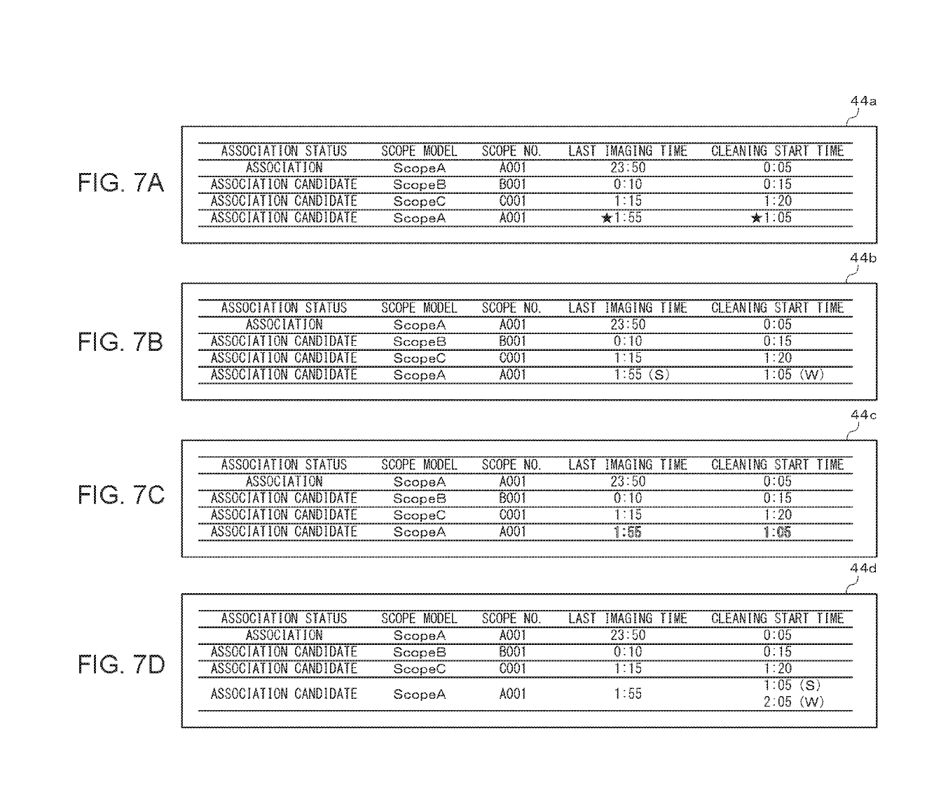

FIGS. 7A to 7D are tables each showing a display example in which examination information including the last imaging time of summer time and cleaning information including a cleaning start time of standard time are displayed on a screen.

In the “association status” on each of the screens 44a to 44d, the “association” shows a status in which examination information and cleaning information have already been associated with each other, while the “association candidate” shows a status in which a suggestion is made to a user. When an agreement operation is made by a user, the “association candidate” is changed to the “association.”

Each of FIGS. 7A and 7B shows an example in which a mark is displayed. On the screen 44a shown in FIG. 7A, a star mark is added to the combination of the last imaging time of summer time and the cleaning start time of standard time. Other combinations are not added with a mark, because the time notations of the last imaging time and the cleaning start time match each other in each of them. On the screen 44b shown in FIG. 7B, a mark of (S) and a mark of (W) are added to the last imaging time of summer time and the cleaning start time of standard time, respectively, in the combination of the last imaging time of summer time and the cleaning start time of standard time.

FIG. 7C shows an example of color classification display. On the screen 44c shown in FIG. 7C, of the combinations of the last imaging time of summer time and a cleaning start time of standard time, the combinations of the last imaging time and a cleaning start time whose time notations match each other are shown with the color classified. FIG. 7D shows an example of two type display. On the screen 44d shown in FIG. 7D, a cleaning start time of summer time is displayed along with a cleaning start time of standard time, in the combination of the last imaging time of summer time and a cleaning start time of standard time.

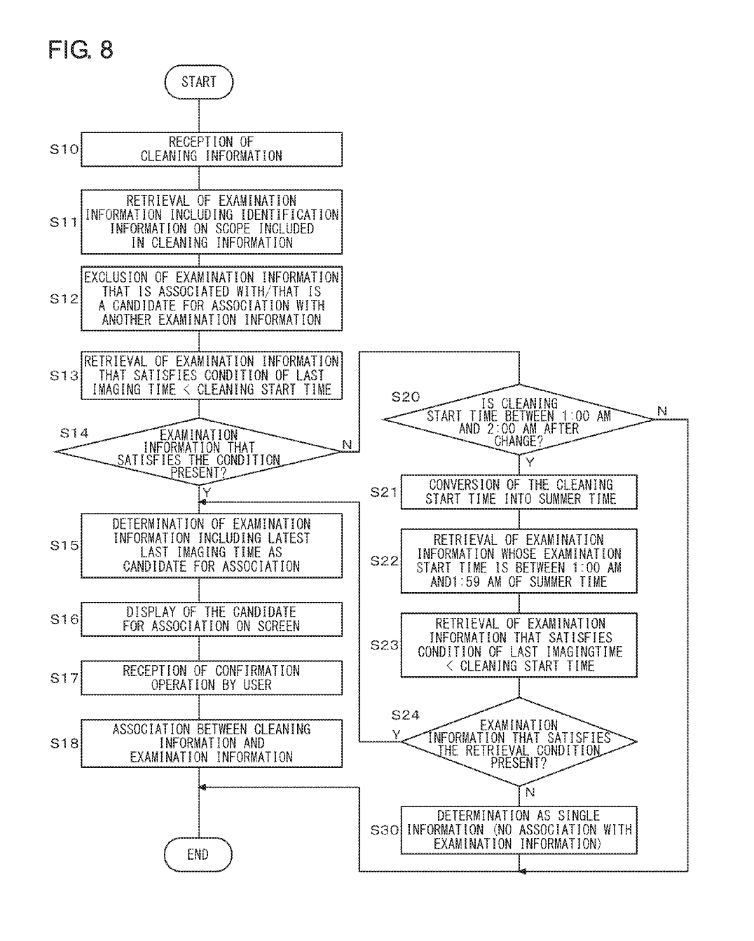

FIG. 8 is a flowchart illustrating a flow of processing according to an embodiment of the invention, in which cleaning information and examination information are associated with each other. The endoscopy service support apparatus 30 receives cleaning information from the cleaning apparatus 50 (S10). The retrieval unit 323 in the endoscopy service support apparatus 30 retrieves examination information including identification information on a scope (e.g., a scope model and a scope serial number) that matches the identification information on a scope included in the cleaning information (S11). The retrieval unit 323 retrieves, of the retrieved examination information, examination information that is not associated with other cleaning information and is not a candidate for the association. That is, cleaning information, which is associated with other cleaning information or which is a candidate for the association with other cleaning information, is excluded (S12). The retrieval unit 323 retrieves, of the examination information that have passed through the processing of Step S12, examination information in which “the last imaging time <a cleaning start time” (S13).

The association candidate determination unit 324 determines whether examination information that satisfies all of the retrieval conditions of Steps S11, S12, and S13 is present (S14). When present (S14/Y), the association candidate determination unit 324 determines, of the examination information that satisfy all of the retrieval conditions, the examination information whose last imaging time is closest to the cleaning start time as a candidate for the association with the cleaning information (S15). The display control unit 326 suggests association between the cleaning information and examination information determined as a candidate for the association by making them displayed on a screen (S16). When receiving a confirmation operation by a user (S17), the association unit 325 associates the cleaning information and the examination information with each other (S18). If association between cleaning information and examination information, both the information never corresponding to each other, is suggested due to a system error, a user can cancel the association between them by performing a denial operation.

When examination information that satisfies all of the retrieval conditions of Steps S11, S12, and S13 is not present in Step S14 (S14/N), the association candidate determination unit 324 determines whether the cleaning start time included in the cleaning information is a time between 1:00 AM to 2:00 AM immediately after the change from summer time to standard time (S20). When the cleaning start time is a time between 1:00 AM and 2:00 AM immediately after the change to standard time (S20/Y), the time conversion unit 327 converts the cleaning start time of standard time into a cleaning start time of summer time (S21). The retrieval unit 323 retrieves examination information whose examination start time is a time between 1:00 AM and 1:59 AM of summer time on the day when summer time is changed to standard time (S22). Information on a date and time of summer time, which is required in the processing of Steps S21 and S22, can be changed by a configuration file. The retrieval unit 323 retrieves, of the examination information that satisfy the condition of Step S22, examination information in which “the last imaging time <a cleaning start time” (S23).

The association candidate determination unit 324 determines whether examination information that satisfies all of the retrieval conditions of Steps S22 and S23 is present (S24). When present (S24/Y), the association candidate determination unit 324 determines, of the examination information that satisfy all of the retrieval conditions, the examination information whose last imaging time is closest to the cleaning start time as a candidate for the association with the cleaning information (S15). The display control unit 326 suggests association between the cleaning information and the examination information determined as a candidate for the association by making them displayed on a screen (S16). When receiving a confirmation operation by a user (S17), the association unit 325 associates the cleaning information and the examination information with each other (S18).

When the cleaning start time included in the cleaning information is not a time between 1:00 AM and 2:00 AM immediately after the change from summer time to standard time, in Step S20 (S20/N), or when examination information that satisfies all of the retrieval conditions of Steps S22 and S23 is not present in Step S24 (S24/N), the association candidate determination unit 324 determines the cleaning information as single cleaning performed independently of examination (S30).

FIGS. 9A and 9B are tables showing examples of examination information and cleaning information that have been associated with each other, respectively. FIG. 9A shows an example of examination information, while FIG. 9B an example of cleaning information. Each examination information is managed by examination information ID, and each cleaning information by cleaning information ID. When examination information and cleaning information are associated with each other, the cleaning information ID of the associated cleaning information is added to the examination information. Similarly, the examination information ID of the associated examination information is added to the cleaning information.

As described above, in basic processing of the present embodiment, examination information including the last imaging time and cleaning information including a cleaning start time are associated with each other by comparing the last imaging time and a cleaning start time to each other, in which: the last imaging time is prior to the cleaning start time; and the cleaning start time is closest to the last imaging time. As exception processing, even if the last imaging time is before 2:00 AM of summer time and the cleaning start time of the scope used in the examination is between 1:00 AM and 2:00 AM of standard time (between 2:00 AM and 3:00 AM of summer time), the association between cleaning information and examination information is suggested, only on the day when the system time returns to standard time from summer time.

In this case, a suggestion, with respect to examination information that satisfies all of the following conditions, is made:

(1) the identification information on a scope included in the examination information matches the identification information on a scope included in the cleaning information;

(2) the examination information is not suggested as a candidate for association with or not associated with other cleaning information; and

(3) the last imaging time of an examination is before 2:00 AM of summer time, and the cleaning start time is between 1:00 AM and 2:00 AM after the change to standard time.

Thereby, when the system time returns to standard time from summer time, it becomes possible to correctly associate examination information and cleaning information with each other, without leaving data that is not associated with. Further, association between examination information and cleaning information, both the information being shifted by one with each other, can be suppressed.

The present invention has been described above based on embodiments. These embodiments are intended to be illustrative only, and it will be obvious to those skilled in the art that various modifications to constituting elements and processes could be developed and that such modifications are also within the scope of the present invention.

Summer time is adopted not only in the U.S.A. but in many European countries. The summer time period in Europe is between 1:00 AM on the final Sunday in March and 1:00 AM on the final Sunday in October. The processing according to the present embodiment can be applied to the processing for associating examination information and cleaning information, either information including 1:00 AM on the final Sunday in October.

What is claimed is:

1. An endoscopy service support apparatus comprising:
an examination information storage unit that stores examination information acquired from an endoscope system; and
a control unit that retrieves, when receiving cleaning information from a cleaning apparatus for cleaning an endoscope, examination information that should be associated with the cleaning information, based on a cleaning start time included in the cleaning information and an examination end time included in examination information stored in the examination information storage unit, wherein
the control unit includes:
a retrieval unit that retrieves examination information, which includes identification information on an endoscope corresponding to identification information on an endoscope included in the cleaning information and in which an examination end time included in the examination information is prior to a cleaning start time included in the cleaning information;
a candidate determination unit that determines, of examination information that satisfy a retrieval condition, examination information including an examination end time closest to the cleaning start time as a candidate for examination information that should be associated with the cleaning information; and
a time conversion unit that converts, when examination information that satisfies the retrieval condition is not present and the cleaning start time is a time included in a period of time corresponding to a time difference between summer time and subsequent standard time, a cleaning start time of standard time included in the cleaning information so as to match the examination end time, based on the time difference between them, and wherein
the retrieval unit re-retrieves examination information based on a conversion result of the time conversion unit.

2. The endoscopy service support apparatus according to claim 1, wherein
when examination information that satisfies the retrieval condition is not present and the cleaning start time is a time included in a period of time corresponding to a time difference between summer time and subsequent standard time, the time conversion unit converts a cleaning start time of standard time included in the cleaning information into a cleaning start time of summer time, and wherein
the retrieval unit re-retrieves examination information by using the converted cleaning start time of summer time.

3. The endoscopy service support apparatus according to claim 1, wherein
the control unit retrieves, each time when the control unit receives cleaning information from the cleaning apparatus, examination information that should be associated with the cleaning information, and wherein
the retrieval unit retrieves examination information: that includes identification information on an endoscope corresponding to identification information on an endoscope included in the cleaning information; in which an examination end time included in the examination information is prior to a cleaning start time included in the cleaning information; and that is not associated with another cleaning information.

4. The endoscopy service support apparatus according to claim 1, wherein
the examination end time included in the examination information is specified by the last imaging time in the examination at which the inside of the body of a patient is imaged with an endoscope.

5. The endoscopy service support apparatus according to claim 1, wherein
the control unit further includes a display control unit that makes a screen displayed, the screen being used for making a user select whether a candidate for examination information determined by the candidate determination unit is to be associated with the cleaning information, and wherein
when displaying, on a screen, examination information including an examination end time of summer time and cleaning information including a cleaning start time of standard time, the display control unit displays them such that a user can recognize a difference between both time notations.
